(19) United States
(12) Patent Application Publication
(10) Pub. No.: US 2025/0041043 A1
(43) Pub. Date: Feb. 6, 2025

(54) IMPLANTABLE DEVICE WITH GRADUAL RELEASE OF ONE OR MORE FUNCTIONAL AGENTS AND METHOD FOR MANUFACTURING SUCH A DEVICE

(71) Applicant: COUSIN BIOTECH, WERVICQ SUD (FR)

(72) Inventor: Stéphane NOEL, HANTAY (FR)

(21) Appl. No.: 18/718,913

(22) PCT Filed: Dec. 14, 2022
(86) PCT No.: PCT/EP2022/085949

§ 371 Date: Jun. 12, 2024

(30) Foreign Application Priority Data
Dec. 15, 2021 (FR) FR2113580

(51) Int. Cl. A61F 2/08

(52) U.S. Cl. CPC A61F 2/08; A61F 2210/0004

(57) ABSTRACT

The present invention relates to an implantable device, in particular for the reconstruction of an anterior cruciate ligament or a tendon, comprising an elongate core (20) and a hollow elongate covering member comprising an inside volume at least partially receiving said elongate core, wherein said elongate core comprises a polymer matrix that is bioerodible in an aqueous medium and one or more functional agents, and the covering member comprises through-holes in fluid communication with said inside volume. The invention also relates to a method for manufacturing such an implantable device.

### TECHNICAL FIELD

The present invention relates to the general field of implantable devices having gradual release of one or more functional agent(s), particularly an osteoinductive and/or osteoconductive agent, for the reconstruction in particular of an anterior cruciate ligament.

### PRIOR ART

The deterioration and in particular the tear of the anterior cruciate ligament, particularly of the knee, is frequent among persons practicing a sports activity. According to studies, this type of injury is reported to affect about 100 000 to 200 000 athletes in the USA per year. In addition to joint instability, the clinical signs can be the manifestation of pain and post-traumatic swelling.

Anatomically, the anterior cruciate ligament (ACL) is a structure connecting the femur to the tibia ensuring different movements in the anterior direction, and inhibits extreme rotation of the knee.

Ligaments are known to be non-regenerative and to have limited vascularization. ACLs therefore have scarce ability to self-heal.

About 150 000 surgical procedures are performed each year in the USA to repair an ACL anomaly and to allow restoring of the normal functions thereof. In general, repair and restoring strategies of ACL functions are based on autografts or allografts such as autograft from the Hamstring tendon and bone autograft from the patellar tendon. Surgical procedure entails the inserting of a graft in a tunnel drilled into a bone element, and the attaching of this graft between the tibia and femur with specific fixation members such as fixation by interference screws. This type of procedure often requires later revision surgery. In addition to risks of comorbidity (infection, reject . . . ) associated with the use of biological grafts, ACL reconstruction can fail for certain problems such as those related to the inter-condylar roof, graft impacts, insertion in a non-anatomic tunnel or the inability to form a transition zone at the interface of graft and bone.

Incomplete healing of the ligament can compromise the success of reconstruction procedure through lack of osseointegration. This lack of osseointegration appears to affect about 15% to 25% of patients treated surgically.

The histological and biomechanical properties of the ACL are ensured by the enthesis, an attachment site contained in native ACL and composed of three regions: fibrocartilage tissue, ligament, and bone. The reproduction of these structures plays a major part in the success of reconstruction of a ligament, and in particular of the ACL.

There is therefore a need for implantable structures formed by interface tissue engineering.

Recently, tissue engineered structures have become the focus of increasing interest and are considered to be a promising alternative in the development of a therapeutic approach to treat bone defects and restore living tissues using biomaterials, cells, and growth factors. Endeavouring to recreate bone morphology is one of the most complex subjects of research on account of the complexity of this bone structure and the property thereof for perpetual remodelling and modification of bone shape. Histologically, the bone is made up of living cells and of an extracellular matrix secreted by osteoblasts. The matrix is composed of two phases, the first phase comprising organic elements essentially containing type 1 collagen (90%) and non-collagenic proteins (10%). This first phase is responsible for bone flexibility and for resistance to twist and tensile forces. The second phase is the mineralized phase composed of 60% of the volume of the extracellular matrix and contains hydroxyapatite (Ca₁₀(PO₄)₆ (OH)₁₂) arranged in crystals and ensuring bone hardness.

The implantable device for bone tissue engineering must be biocompatible, optionally fully or partially biodegradable, must promote osseointegration and must have sufficient mechanical strength to withstand tensile and/or rotational loads to which a tendon or ligament can be subjected.

Additionally, a structure in a hydrophobic synthetic material for implantable devices e.g. in polyethylene terephthalate tends to inhibit tissue adhesion.

There is therefore a need for an implantable device formed by bone tissue engineering which solves the aforementioned problems.

### DESCRIPTION OF THE INVENTION

The subject of the present invention is advantageously an implantable device for bone tissue engineering, in particular for the reconstruction of a ligament or tendon connected to a bone element, the device having a structure reproducing and/or imitating the extracellular bone matrix and promoting bone formation i.e. promoting osteoconduction.

Advantageously the subject of the present invention is an implantable device promoting osseointegration, mechanically imitating the behaviour of a ligament or tendon, and promoting cell adhesion.

In a first aspect, the subject of the present invention is an implantable device remedying all or some of the aforementioned problems, in particular for the reconstruction of an anterior cruciate ligament or a tendon, comprising an elongate core and a hollow elongate covering member comprising an inside volume at least partially receiving said elongate core. Advantageously, said elongate core comprises a polymer matrix bio-erodible in an aqueous medium, and one or more functional agents, and the covering member comprises through-holes in fluid communication with said inside volume.

Advantageously, the bio-erodible polymer matrix is arranged in the inside volume of the hollow member so that gradual disintegration thereof, in particular gradual solubilization thereof, is slowed by the structure of the hollow elongate member.

In addition, the combination of an elongate core and of the hollow elongate member allows the imparting of mechanical strength properties against tensile and torsion forces, that are sought after to reproduce the properties of a tendon or ligament.

Advantageously, the hollow elongate member also allows immobilization of the functional agent(s) within the inside volume in a manner that is controlled according to the chosen arrangement of the through-holes.

This particular provision of the device provides control over the gradual release of the functional agent(s).

Gradual release is therefore controlled first by the bio-erodible nature of the matrix in an aqueous medium, but also by the mechanical barrier of the hollow member the structure of which is determined to allow first the release of the functional agent(s) through the through-holes and secondly to allow the formation of bone on the outer surface thereof.

By implantable device in the present invention, it is meant any device configured to be implanted in a human or animal body for a period of several days to several months or several years.

### Bio-Erodible Polymer Matrix

By bio-erodible herein, it is meant the degradation, disassembling or digestion of the polymer matrix, and in particular of the bio-resorbable polymer(s) included in the matrix, via the action of one or more environmental biological factor(s) (e.g. acidity, temperature or humidity of the target site, the presence of enzyme(s), protein(s) or other molecule(s) at the target site), or via the action of physical or chemical properties of the functional agent(s) dispersed in the matrix, preferably via the action of at least one aqueous medium, more preferably via the action of water contained in the medium of the implantation site.

By polymer matrix, it is meant herein a physical structure of polymer(s) which retain one or more functional agent(s).

Advantageously, the polymer matrix comprises one or more bio-resorbable polymer(s), particularly having a determined solid structure before being contacted with an aqueous medium.

Preferably, by the term “polymer” it is meant herein a polymer or oligomer having a repeating unit, or two or three or more repeating units differing from each other.

Therefore, by the term copolymer herein, it is meant any polymer or oligomer having two or more repeating units differing from each other.

In particular an oligomer herein comprises a number of repeating units less than or equal to 10, and a polymer has a number of repeating units higher than 10.

By bio-resorbable (or resorbable), it is meant to be understood herein any material which, when placed in a living body, will disappear after a determined length of time, for example after three days, or after 3 months, or 6 months, or longer.

The choice of bio-resorbable material is within the reach of persons skilled in the art and is dependent on the degree of polymerization and/or degree of crosslinking and/or chemical nature thereof to obtain resorption after a determined length of time.

Advantageously, the implantable device is an artificial ligament or artificial tendon, in particular one that is osteoconductive and/or osteoinductive.

Advantageously the polymer matrix is water-soluble i.e. that in contact with water, in particular in contact with a physiological medium, said matrix gradually dissolves, for example said matrix is fully dissolved after a time of 3 months or 6 months or longer depending on needs.

In particular, the polymer matrix is water-soluble in contact with the aqueous medium of the region of the living body in which it is implanted.

In particular, the polymer matrix is water-soluble in contact with a medium comprising water and having a temperature higher than or equal to 20° C., in particular higher than or equal to 25° C., or 30° C. or 35° C. or 37° C. Preferably, the aqueous medium comprises at least 50% or 60% or 70% or 80% or 90% by weight or by volume of water, in particular distilled and/or reverse osmosis water.

The aqueous medium can be a physiological medium, in particular an aqueous medium comprising distilled water and sodium chloride (e.g. diluted in a proportion of 0.9% by weight of NaCl relative to the volume of the medium) and optionally other minerals such as potassium chloride, calcium chloride, magnesium sulfate or a mixture of the latter.

Advantageously, one or more functional agent(s), in particular the functional agent(s), are dispersed in the bio-erodible polymer matrix.

Preferably, the bio-erodible polymer matrix comprises the functional agent(s).

Preferably the mass of the bio-erodible polymer matrix and of one or more functional agent(s) is higher than or equal to 0.05 g/metre of implantable device and less than or equal to 10 g/metre of implantable device, more preferably less than or equal to 5 g/metre or 3 g/metre or 2 g/metre or 1 g/metre.

Preferably, the overall structure of the implantable device is elongate and is of a determined length (cm).

### Functional Agent(s)

Advantageously, by functional agent it is understood herein any agent having a bone integrating function, in particular osteoconductive and/or osteoinductive, and/or any agent having a prophylactic function and/or a curative function of a given pathology.

By osteoinductive functional agent, it is understood herein any agent allowing the inducing the differentiation of stem cells into bone cells, in particular without any immunity reaction.

By osteoconductive functional agent, it is understood herein any functional agent promoting bone formation.

### Elongate Core

Advantageously, by elongate core herein it is meant any element having a length distinctly greater than the thickness or width thereof, for example a length greater by at least 5 or 8 or 10 or 15 or 20 or 30 times the width or thickness of said elongate core.

The elongate core is preferably a textile elongate core.

The elongate core may comprise (or can be) one or more twisted yarn(s), core spun yarn(s), or braided yarn(s), for example it is a braid in particular a solid braid; a narrow knitted or woven strip, for example it can be a knitted tube; or a strip in one or more nonwoven(s), in particular heat-bonded.

Preferably the textile elongate core comprises one or more yarn(s) and/or fibres.

Preferably, the elongate core is non-resorbable.

By non-resorbable herein it is meant any material or structure which, when implanted in a living body, will not resorb i.e. will remain there for several years without any significant degradation of structure.

The textile elongate core is preferably a braid, in particular a solid braid (i.e. not hollow).

The elongate core preferably has a diameter less than or equal to 20 mm or 15 mm or 10 mm or 8 mm or 5 mm.

The elongate core preferably has a diameter greater than or equal to 1 mm or 2 mm or 3 mm.

The elongate core preferably has a length greater than or equal to 10 mm or 20 mm or 30 mm or 40 mm or 50 mm or 80 mm.

The elongate core preferably has a length less than or equal to 150 or 130 mm or 110 mm or 90 m or 70 mm or 50 mm or 40 mm or 30 mm.

Advantgaeously, the outer surface of the elongate core is directly oriented facing the inner surface of the hollow elongate member.

Advantageously, the elongate core has a mass per unit length greater than 0 g/linear metre and less than or equal to 10 g/linear metre, or 8 g/linear metre, or 5 g/linear metre, or 4 g/linear metre, or 3 g/linear metre, or 2 g/linear metre or 1 g/linear metre, for example it is between 0.50 g/linear metre and 0.80 g/linear metre.

Advantageously, the elongate core has a porous surface i.e. comprising openings optionally through-holes. The pores are advantageously formed by the interstices between the yarn(s) and/or fibres.

In one embodiment, the elongate core comprises between 8 and 20 textile strands, in particular braided strands, in particular each textile strand comprises one or more yarns.

In one embodiment, the elongate core (in particular the auxiliary textile covering) comprises between 12 and 18 textile strands (for example about 16 textile strands), that in particular are braided, and each textile strand particularly comprises one or more yam(s).

Preferably the textile elongate core comprises several multifilament yarns, more preferably each having a titer higher than or equal to 50 dtex and lower than or equal to 500 dtex or 350 dtex or 300 dtex.

Preferably, the textile elongate core, braided in particular, comprises a first multifilament yarn having a titer T1 (dtex) and a second multifilament yarn having a titer T2 (dtex), the ratio T2/T1 being higher than or equal to 1.5; in particular higher than or equal to 2.

Preferably, the textile elongate core comprises:
an auxiliary core comprising one or more yarn(s) (in particular two or three yarns), in particular multifilament yarns, more particularly at least one of said yarns (and particularly three) having a titer (dtex) ranging from 100 dtex or 150 dtex to 300 dtext or 250 dtex; and
an auxiliary textile covering, especially braided, around said auxiliary core, in particular comprising one or more multifilament yarn(s), and more particularly comprising said first and second multifilament yarns described in the preceding paragraph.

Advantageously, the auxiliary core comprises one or more non-twisted multifilament yarn(s) or arranged substantially parallel to each other.

### Hollow Elongate Covering Member

Advantageously by hollow elongate covering member it is meant herein a covering member having a length distinctly greater than the thickness or width thereof, for example a length greater than at least 5 or 8 or 10 or 15 or 20 or 30 times the width or thickness of said covering member.

The hollow elongate member is preferably a textile hollow elongate member.

Preferably, the hollow elongate covering member and the elongate core are arranged coaxially, in particular the respective central longitudinal axes thereof substantially merge.

In general, by textile herein it is meant any element (core, hollow member . . . ) obtained by handling one or more yarn(s) and/or fibres.

Preferably, the hollow elongate covering member is a textile elongate member and may comprise (or can be) a knitted tube; a hollow braid; or a tube in one or more nonwoven(s), heat bonded in particular.

Preferably the textile hollow elongate member comprises one or more yarn(s) and/or fibres.

Preferably the hollow elongate member is non-resorbable.

The textile elongate member is preferably a hollow braid.

The hollow elongate member preferably has an outer diameter of less than or equal to 20 mm or 15 mm or 10 mm or 8 mm or 5 mm.

The hollow elongate member preferably has an outer diameter greater than or equal to 1 mm or 2 mm or 3 mm.

The hollow elongate preferably has a length longer than or equal to 10 mm or 20 mm or 30 mm or 40 mm or 50 mm or 80 mm.

The hollow elongate member preferably has a length shorter than or equal to 150 mm or 130 mm or 110 mm or 90 mm or 70 mm or 50 mm or 40 mm or 30 mm. Advantageously, the hollow elongate covering member has an inner surface directly oriented facing the outer surface of the elongate core.

Advantageously, the hollow elongate covering member has an outer surface substantially opposite the inner surface thereof.

Preferably, the thickness of the wall of the hollow elongate member extends between the inner surface to the outer surface thereof.

Advantageously, the hollow elongate covering member has a mass per unit length greater than 0 g/m² and less than or equal to 10 g/linear metre or 8 g/linear metre or 5 g/linear metre or 4 g/linear metre or 3 g/linear metre or 2 g/linear metre or 1 g/linear metre, for example it is between 0.50 g/linear metre and 0.90 g/linear metre.

Advantageously, the hollow elongate covering member comprises through-holes extending in particular between the inner surface and outer surface thereof, in particular opening onto said inner and outer surfaces of the hollow elongate covering member.

The pores are advantageously formed by the interstices between the yarn(s) and/or the fibres. These openings allow the placing in fluid communication of the inside volume of the elongate member with the outside and hence the outer surface thereof. Therefore, the functional agent(s) contained in the bio-erodible polymer matrix are released, as and when the matrix degrades, into the inside volume and then onto the outer surface of the elongate member. The outer surface of the elongate member therefore advantageously allows the forming of an osseointegration outer surface.

Preferably, the hollow elongate member is over-knit or over-braided, preferably over-braided around the elongate core.

In one embodiment, the elongate member comprises between 8 and 20 textile strands, braided in particular, and each textile strand particularly comprises one or more yarn(s).

In one embodiment, the elongate member comprises between 10 and 18 braided textile strands (in particular 12 or 16 braided strands), in particular each textile strand comprises one or more yarn(s), more particularly between 3 and 8 yarns.

Preferably, the hollow elongate member, braided in particular, comprises several multifilament yarns (braided in particular), more preferably between 30 and 75 or 70 multifilament yarns, preferably between 40 and 70 multifilament yarns, in particular between 50 and 70 multifilament yarns or between 55 and 70 multifilament yarns, or between 60 and 68 multifilament yarns, each yarn having a titer higher than or equal to 50 dtex or 80 dtex or 100 dtex and lower than or equal to 300 dtex or 250 dtex or 200 dtex or 150 dtex.

### Through-Holes

The through-holes can be of any shape, and can be ovoid or polygonal in particular.

If the through-holes are ovoid, the openings are preferably circumscribed within a circle of diameter 20 μm to 5000 μm, more preferably of diameter 20 μm to 500 μm.

If the through-holes are polygonal, the lengths of the sides of the polygons are preferably longer than or equal to 20 μm and shorter than or equal to 5000 μm, more preferably longer than or equal to 20 μm and shorter than or equal to 500 μm.

### Yarn(s)/Fibre(s)

The yarn(s) (in particular of the elongate core and/or of the covering member and/or of the auxiliary elongate core and/or of the auxiliary covering member) can be a monofilament yarn, a spun fibre yarn or multifilament yarn.

The yarn(s) and/or fibres may comprise a material (or be in a material) chosen from among: polyesters in particular polyethylene terephthalate (PET) e.g. Dacron®, and polybutylene terephthalate (PBT); polyamides such as PA 6-6, PA 6, PA 4-6; polyolefins such as polypropylene, polyethylene (in particular low density, or high or very high density); polymers of lactic acid and optionally of glycolic acid such as PLLA, PLDA and PLGA.

The yarn(s) and/or fibres can be bioresorbable, non-bioresorbable or semi-bioresorbable, preferably non-resorbable.

Advantageously, the multifilament yarn comprises a number of filaments of between 5 and 30, in particular between 5 and 20 filaments.

Advantageously, a filament of a multifilament yarn has a mass per unit length of between 1 dtex and 30 dtex, preferably between 5 dtex and 20 dtex.

Preferably, the yarn(s) comprise at least one material chosen from among: polyesters, in particular polyethylene terephthalate (PET) e.g. Dacron®, or polybutylene terephthalate (PBT); or polyolefins such as polypropylene, polyethylene (in particular low density, or high or very high density); or a mixture thereof.

Preferably, the yarn(s) are in polyethylene terephthalate (PET) e.g. Dacron® or in polybutylene terephthalate (PBT); or in polypropylene or in polyethylene; or a mixture thereof (e.g. one or more yarn(s) are in PET or PBT and one or more yarn(s) are in PP or PE).

Advantageously, the bio-erodible polymer matrix forms a polymer coating, at least partially coating the outer surface of the elongate core, in particular the outer surface of the auxiliary textile covering.

Advantageously, the bio-erodible polymer matrix fully impregnates the elongate core.

In particular, by “fully impregnates” it is meant that the elongate core comprises the polymer matrix at least partially coating the auxiliary textile core and the auxiliary textile covering.

In one variant, the textile elongate core comprising in particular an auxiliary textile core and an auxiliary textile covering which is at least partially arranged around said auxiliary textile core, and the textile hollow covering member together form a textile assembly having a total titer higher than or equal to 18 000 dtex and lower than or equal to 25 000 dtex, preferably lower than or equal to 24 000 dtex, more preferably lower than or equal to 22 000 dtex, in particular higher than or equal to 20 000 dtex, more particularly higher than or equal to 21 000 dtex.

In one variant, the functional agent(s) comprise one or more osteoinductive and/or osteoconductive agent(s).

In one variant, the osteoinductive and/or osteoconductive agent(s) are chosen from among: bioglass; calcium phosphate bioceramics in particular hydroxyapatites; tricalcium phosphate and a mixture thereof.

Advantageously, the osteoinductive and/or osteoconductive agent(s), or the osteoinductive and/or osteoconductive agent(s), comprise one or more osteoinductive and/or osteoconductive agent(s) chosen from among bioglass materials.

Advantageously, the osteoinductive and/or osteoconductive agent(s), or the osteoinductive and/or osteoconductive agent(s), comprise one or more osteoinductive and/or osteoconductive agent(s) chosen from among calcium phosphate-based bioceramics, in particular hydroxyapatites, and tricalcium phosphate or a mixture thereof.

Preferably, the hydroxyapatite is a calcium phosphate belonging to the apatite family. This is a generic name designating phosphates of varying composition.

Preferably the bioglass is 45S5 bioglass. Advantageously, this bioglass is osteoconductive and osteoinductive.

Preferably, the bioglass is a bioceramic comprising silica, sodium, calcium, and phosphate, in particular in varying amounts, more particularly the weight fraction of silica (in particular SiO₂) is in majority relative to the other components.

The osteoinductive and/or osteoconductive agent(s) are water-soluble or non-water-soluble, preferably water-soluble.

Preferably, the osteoinductive and/or osteoconductive agent(s) are porous.

Preferably, the bioglass comprises:
from 30 weight % to 55 weight % of SiO₂, in particular 40 weight % to 50 weight % of SiO₂; and/or
from 15 weight % to 35 weight % of Na₂O, in particular 20 weight % to 30 weight % of Na₂O; and/or
from 15 weight % to 35 weight % of CaO, in particular 20 weight % to 30 weight % of CaO; and/or
from 2 weight % to 15 weight % of P₂O₅, in particular from 4 weight % to 10 weight % of P₂O₅.

In one variant, the functional agent(s) are chosen from among: anti-inflammatories, antibiotics, antibacterials, analgesics, and a mixture thereof.

In one variant, the bio-erodible polymer matrix comprises one of more functional agent(s) which are osteoinductive and/or osteoconductive agent(s), and optionally one or more functional agent(s) chosen from among: anti-inflammatories, antibiotics, antibacterials, analgesics, and a mixture thereof.

In one variant, the elongate core comprises a textile elongate core, in particular a braid.

In one variant, the textile elongate core is coated at least partially by the bio-erodible polymer matrix in which the functional agent(s) are dispersed.

The coating of bio-erodible polymer matrix can be continuous, for example in the form of a film, or discontinuous for example arranged in dots, strips, varied patterns, or a combination thereof.

Advantageously, the elongate core is fully or partially coated on the outer surface thereof by the bio-erodible matrix comprising one or more functional agent(s).

In one variant, the hollow elongate covering member comprises a textile elongate covering member, in particular it comprises a hollow braid.

Advantageously, the covering member is over-braided around the elongate core so that the assembly is perfectly homogeneous and has good cohesion.

In one variant, the bio-erodible polymer matrix comprises a polymer or several mixed polymers that are dehydrated and able to form a gel soluble in an aqueous medium.

Advantageously, said dehydrated polymer(s) are bio-erodible polymers, in particular bio-resorbable.

Advantageously, the polymer matrix is solid at ambient temperature i.e. at a temperature higher than or equal to 0° C. and lower than or equal to 40° C. or 37° C. or 30° C., the polymer matrix comprising at most 10% or 5% by weight of water relative to the total weight thereof (comprising the functional agent(s)).

When the polymer matrix is in contact with an aqueous medium, the matrix absorbs water, may optionally swell depending on the polymer used, then gradually dissolves releasing the dispersed functional agent(s) contained therein.

By hydrated polymer, it is meant herein any polymer able to form a gel in an aqueous medium and then progressively to solubilize. The polymer is not in gel form before implantation. It is therefore dehydrated.

In one variant, the bio-erodible polymer matrix comprises one or more bio-resorbable polymers chosen from among the following polymers: (co)polymers of cyclodextrin(s) and/or of derivative(s) of cyclodextrin(s) and/or of inclusion complex(es) of cyclodextrin(s) and/or of derivatives of soluble cyclodextrin(s); a polysaccharide derivative whether or not crosslinked such as collagen polymers; polymers derived from hyaluronic acid; polymers of carboxymethylcellulose or derivatives of carboxymethylcellulose; vinyl polymers such as polyvinylpyrrolidone polymers (PVP) or polyvinylpolypyrrolidone polymers (crospovidone); polyethylene glycol polymers; lactic acid polymers; copolymers of lactic acid and polyethylenegylcol (PLLA/PEG); copolymers of lactic acid, glycolic acid and polyethyleneglycol (PLGA/PEG); polymers of polyvinyl alcohols (PVA); and polymers of polyacrylates such as polyhydroxyethylmethacylate (PHEM); and a mixture thereof.

In one embodiment, the bio-erodible polymer matrix comprises one or more bio-resorbable polymer(s) chosen from among vinyl polymers such as polymers of polyvinylpyrrolidone (PVP) or polymers of polyvinylpolypyrrolidone (crospovidone), or a mixture of thereof.

In one embodiment, the bio-erodible polymer matrix comprises one or more bio-resorbable polymer(s) chosen from among: (co)polymers of cyclodextrin(s) and/or derivative(s) of cyclodextrin(s) and/or inclusion complex(es) of cyclodextrin(s) and/or derivatives of soluble cyclodextrin(s); or a mixture thereof.

In one embodiment, the bio-erodible polymer matrix comprises one or more bio-resorbable polymer(s) chosen from among:
a polysaccharide derivative whether or not crosslinked such as collagen polymers; or
polymers derived from hyaluronic acid; or
polymers of carboxymethylcellulose or derivatives of carboxymethylcellulose; or
a mixture thereof.

The bio-resorbable polymer(s) of the bio-erodible polymer matrix are preferably water-soluble.

The (co)polymers of cyclodextrin(s) and/or of derivative(s) of cyclodextrin(s) and/or of inclusion complex(es) of cyclodextrin(s) are preferably water-soluble.

These (co)polymers can be obtained 1/by preparing in the solid state a mixture of cyclodextrin(s) and/or derivative(s) of cyclodextrin(s) and/or inclusion complex(es) of cyclodextrin(s) and/or derivatives of cyclodextrin(s), with a poly(carboxylic) acid or anhydride of poly(carboxylic) acid or a mixture of poly(carboxylic) acids and/or anhydrides of poly(carboxylic) acids and optionally a catalyst; and 2/heating said solid mixture to a temperature of between 100° C. and 200° C. for a time of between 1 min and 60 min, preferably of 30 min.

Preferably, to prepare the mixture in the solid state, an aqueous solution is prepared of cyclodextrin(s) and/or derivatives of cyclodextrin(s) and/or inclusion complex(es) of cyclodextrin(s) and/or derivatives of cyclodextrin(s), and a poly(carboxylic) acid and/or an anhydride of poly(carboxylic) acid and/or a mixture of poly(carboxylic) acids and/or of anhydrides of poly(carboxylic) acids, and optionally a catalyst, and the water of said solution is evaporated at a temperature of between 40° C. and 100° C., preferably under a vacuum at 90° C.

Preferably, the catalyst is chosen from among dihydrogen phosphates, hydrogen phosphates, phosphates, hypophosphites, phosphites of alkali metals, polyphosphoric acid alkali metal salts, carbonates, bicarbonates, acetates, borates, alkali metal hydroxides, aliphatic amines and ammonia, and preferably from among sodium hydrogen phosphate, sodium dihydrogen phosphate and sodium hypophosphite.

Preferably, the cyclodextrin is chosen from among α-cyclodextrin, β-cyclodextrin and γ-cyclodextrin, and the derivatives of cyclodextrin are chosen from among methylated or acetylated hydroxypropyl derivatives of α-cyclodextrin, of β-cyclodextrin and of γ-cyclodextrin, and the inclusion complexes of said cyclodextrins and said derivatives of cyclodextrin(s).

Preferably, the poly(carboxylic) acid and anhydride of poly(carboxylic) acid are chosen from among the following polycarboxylic acids and anhydrides of poly(carboxylic) acids: saturated and unsaturated acyclic poly(carboxylic) acids, saturated and unsaturated cyclic poly(carboxylic) acids, aromatic poly(carboxylic) acids, hydroxypoly(carboxylic) acids, preferably from among citric acid, poly(acrylic) acid, poly(methacrylic) acid, 1,2,3,4-butane tetracarboxylic acid, maleic acid, citraconic acid, itaconic acid, 1,2,3-propane tricarboxylic acid, aconitic acid, all-cis-1,2,3,4-cyclopentanetetracarboxylic acid, mellitic acid, oxydisuccinic acid, thiodisuccinic acid.

Preferably, the poly(carboxylic) acid is chosen from among saturated or unsaturated acyclic poly(carboxylic) acids, saturated and unsaturated cyclic poly(carboxylic) acids, aromatic poly(carboxylic) acids, hydroxypropyl(carboxylic) acids, preferably from among citric acid, poly(acrylic) acid, poly(methacrylic) acid, 1,2,3,4-butanetetracarboxylic acid, 1,2,3-propane tricarboxylic acid, aconitic acid, all-cis-1,2,3,4-cyclopentanetetracarboxylic acid, mellitic acid, oxydisuccinic acid, thiodisuccinic acid.

In one variant, the bio-erodible polymer matrix comprises one or more polymer(s) chosen from among: polymers of polyvinylpyrrolidone (PVP); polymers of polyvinyl alcohol; (co)polymers of cyclodextrin(s) and/or derivative(s) of cyclodextrin(s) and/or inclusion complex(es) of cyclodextrin(s), in particular water-soluble, or a mixture of thereof.

In one variant, the bio-erodible polymer matrix comprises one or more bio-erodible polymer(s), and the weight ratio (functional agent(s)/bio-erodible polymer(s)) in the polymer matrix, in particular the weight ratio (osteoinductive and/or osteoconductive agent(s)/bio-erodible polymer(s)) in the polymer matrix is higher than or equal to 0.20 and lower than or equal to 1.8; preferably higher than or equal to 0.20 and lower than or equal to 1; more preferably higher than or equal to 0.40 and lower than or equal to 0.80; further preferably higher than or equal to 0.50 and lower than or equal to 0.70.

In one variant, the hollow elongate covering member comprises a braid comprising from 8, in particular from 12 to 30, braided strands, and each braided strand comprises from 2 to 8 yarns, preferably said yarns each have a titer ranging from 80 dtex to 200 dtex.

Preferably, each braided strand comprises from 3 to 8 yarns, in particular from 3 to 7 yarns, more particularly from 3 to 6 yarns, optionally from 5 to 7 yarns, for example 4 yarns.

Preferably the covering member comprises from 20 to 28 braided strands.

Preferably the covering member comprises from 8 to 18 braided strands, in particular 10 to 18 braided strands, more particularly 12 to 16 braided strands, for example 12 braided strands or 16 braided strands.

With this particular structure, it is possible to obtain through-holes having optimized dimensions for the gradual migration of the functional agent(s) onto the outer surface of the covering member, and in particular the formation of an osseointegration outer surface if there are one or more osteoinductive and/or osteoconductive functional agents.

In one variant, the hollow elongate covering member comprises between 30 and 120 yarns, preferably between 30 and 80 yarns, in particular said yarns are not bioresorbable.

Preferably, the braided hollow elongate covering member comprises 40 braided yarns to 75 braided yarns, more preferably 45 braided yarns to 68 braided yarns, for example 60 braided yarns to 68 braided yarns.

Preferably, at least one of said yarns is a multifilament yarn, in particular said yarns are multifilament yarns.

The covering member affords good mechanical performance whilst providing deformability on account of the high number of multifilament yarns.

In one variant, the mass fraction of the bio-erodible polymer matrix comprising the functional agent(s) in said implantable device is higher than or equal to 5% and lower than or equal to 25%, preferably higher than or equal to 10% and lower than or equal to 20%.

Advantageously, the mass fraction of the bio-erodible polymer matrix and functional agent(s) is measured relative to the total mass of the implantable device, in particular comprising the polymer matrix, the functional agent(s), the elongate core, and the hollow elongate covering member.

In one variant, the mass fraction of the elongate core (in particular the elongate core alone) free of the polymer matrix and functional agent(s) and of the hollow elongate covering member (in particular the bare hollow elongate covering member) measured relative to the total mass of the implantable device is higher than or equal to 50% or 60% or 70%, preferably higher than or equal to 75%.

In a second aspect, the subject of the invention is a method for manufacturing an implantable device, in particular according to any of the variants of embodiments with reference to the first aspect of the invention, advantageously comprising:
a) a step to apply a bio-erodible polymer coating comprising one or more functional agent(s) onto an elongate, preferably textile, core;
b) a step, in particular by over-braiding, to arrange a hollow elongate covering member, preferably textile member, around at least part of said preferably textile elongate core that is at least partially coated;
c) a step to recover an implantable device comprising an elongate core and a hollow elongate covering member having an inside volume at least partially receiving said elongate core, said elongate core comprises a polymer matrix bio-erodible in an aqueous medium, and one or more functional agent(s), and the covering member comprises through-holes in fluid communication with said inside volume.

In one embodiment, the application step a) comprises a step (a1) to apply a liquid preparation comprising one or more bio-erodible (co)polymers, or one or more precursor monomers and/or oligomers of said (co)polymers bio-erodible in an aqueous medium, and also comprising one or more functional agents, onto an elongate preferably textile core, and a drying step (a2), optionally followed by a polymerization step (a3).

The application step a) may comprise an impregnation step (a1) or spray step (a1) or a soaking step (a1) with a solution/dispersion comprising the (co)polymer(s) and/or precursor monomer(s) and/or oligomer(s) of said polymer(s), and optionally a mangling step (a11) e.g. a calendering step (a11). Step (a1) and/or step (a11) can be performed several times, for example at least 4 times or at least 8 times according to the desired take-up rate.

The liquid preparation can be an aqueous solution/dispersion or a solvated solution/dispersion (differing from water) or a solution/dispersion based on at least one alcohol e.g. ethanol-based.

Preferably the mass faction of bio-erodible (co)polymer(s) or precursor monomer(s) or oligomer(s) of said bio-erodible (co)polymer(s), in the liquid preparation (mass/volume) is higher than or equal to 5% and lower than or equal to 30%, in particular higher than or equal to 10% and lower than or equal to 25%.

The drying step (a2) preferably comprises heating the elongate core at least partially coated with the liquid preparation, to evaporate the water and/or one or more solvent, for example the heating temperature is between 70° C. and 100° C. or 90° C. or 80° C. for 30 minutes or longer until the formation of a solid bio-erodible polymer matrix.

The partially coated elongate core can then be heated at a polymerization step (a3) at which the partially coated elongate core is heated to polymerize, optionally crosslink the precursor monomer(s) or oligomer(s) of said bio-erodible (co)polymer(s), for example to a temperature of 150° C. for at least 30 minutes for (co)polymers of cyclodextrins.

In one variant, step b) is a braiding step of a hollow braid on a braiding device (e.g. a braiding machine) having a total number of spindles higher than or equal to 12, more particularly higher than or equal to 18 and lower than or equal to 30, in particular lower than or equal to 26, and the total number of empty spindles is higher than or equal to 10% and lower than or equal to 60% of the total number of spindles, in particular higher than or equal to 20% and lower than or equal to 60% of the total number of spindles, in particular the number of filled spindles is higher than or equal to 40% and lower than or equal to 90% of the total number of spindles on the braiding device.

Advantageously, the number of filled spindles is higher than or equal to 40%, in particular higher than or equal to 45% and lower than or equal to 90%, in particular lower than or equal to 80% more particularly lower than or equal to 70% of the total number of spindles on the braiding loom.

In one embodiment, the number of spindles on the braiding loom at step b) to over-braid the covering member is between 20 and 26, in particular about 24 spindles.

In one embodiment, the number of filled spindles is between 60% and 75% of the total number of spindles on the braiding loom at step b).

In one embodiment, the number of filled spindles is between 10 and 18, in particular between 12 and 18, and more particularly between 14 and 18.

By filled spindle of a braiding loom it is meant herein that this spindle carries one or more yarn(s) when braiding, and by empty spindle of a braiding loom it is meant that this spindle does not carry any yarn during braiding.

This arrangement of filled and empty spindles on the braiding loom allows spacing of the braided strands and the forming of through-holes promoting gradual migration and fixing of functional agent(s) on the outer surface of the hollow elongate covering member.

Preferably the number of spindles (filled and/or empty) is an integer.

In one variant, step b) is a step to braid a hollow braid on a braiding device comprising filled spindles and empty spindles, and the filled spindles are distributed in at least three groups of filled spindles (in particular in four groups of filled spindles), each group comprising several adjacent filled spindles (in particular two or three or four adjacent filled spindles) and a group of filled spindles is separated from another group of filled spindles by at least one empty spindle, preferably by at least two or three empty spindles.

Advantageously a group of filled spindles comprises at least three or four adjacent filled spindles.

Preferably, the braiding device comprises 10 to 30 spindles, more preferably 14 to 28 spindles, in particular 20 to 28 spindles, more particularly 22 to 26 spindles.

In one embodiment, the braiding device comprises 4 groups of three or four adjacent filled spindles, and 4 groups of 3 or 4 adjacent empty spindles, a group of adjacent filled spindles being arranged between two groups of empty spindles.

In one variant, the braiding device comprises three or four groups of three or four adjacent filled spindles and three or four groups of adjacent empty spindles, a group of adjacent filled spindles being arranged between two groups of adjacent empty spindles.

In one variant, step b) is a braiding step of a hollow braid having a substantially constant braiding angle greater than or equal to 2 mm and smaller than or equal to 15 mm, preferably greater than or equal to 4 mm and smaller than or equal to 10 mm. Advantageously the braiding angle can be measured under a microscope.

A further subject of the present invention, in a third aspect, is an implantable device able to be obtained with the manufacturing method according to the second aspect of the invention.

In particular, the variants/embodiments/definitions of the first aspect of the invention apply independently of each other to the implantable device according to the third aspect and to the manufacturing method according to the second aspect of the invention.

### DESCRIPTION OF THE DRAWINGS

The present invention will be better understood on reading the following nonlimiting embodiments illustrated in the Figures in which:

FIG. 1 schematically illustrates a first example of arrangement of the filled spindles with the empty spindles on a braiding loom comprising a total number of 16 spindles, for the braiding of a first example of hollow elongate covering member according to the invention.

FIG. 2 schematically illustrates a second example of arrangement of the filled spindles with the empty spindles on a braiding loom comprising a total number of 16 spindles, for the braiding of a second example of hollow elongate covering member according to the invention.

FIG. 3 schematically illustrates a third example of arrangement of the filled spindles with the empty spindles on a braiding loom comprising a total number of 24 spindles, for the braiding of a third example of hollow elongate covering member according to the invention.

FIG. 4 schematically illustrates a fourth example of arrangement of the filled spindles with the empty spindles on a braiding loom comprising a total number of 24 spindles, for the braiding of a fourth example of hollow elongate covering member according to the invention.

FIG. 5 is a photograph obtained under scanning electron microscope of the outer surface of a first example of elongate core according to the invention at least partially coated with a bio-erodible matrix comprising at least one osteoinductive and/or osteoconductive functional agent after 7 days' immersion in a simulated body fluid for the study of bio-mineralization.

FIG. 6 is a photograph obtained under scanning electron microscope of the outer surface of the third example of covering member according to the invention after 7 days' immersion in a simulated body fluid to study bio-mineralization.

FIG. 7 is a graph showing the mass proportion of calcium (Ca) and phosphorus (P) along the Y-axis, measured by Energy Dispersive X-ray spectroscopy, on the outer surface of an example of elongate core of the invention referenced (IB) on the X-axis, on the inner surface of a third example of covering member of the invention referenced (IF) on the X-axis, and on the outer surface of the third example of covering member of the invention referenced (EF)-after 7 days' immersion in a simulated body fluid for the study of bio-mineralization.

FIG. 8 is a photograph obtained under scanning electron microscope of the outer surface of the fourth example of covering member according to the invention after 7 days' immersion in a simulated body fluid for the study of bio-mineralization.

### DESCRIPTION OF EMBODIMENTS

### A-Preparation of a Polymer Solution

About 4.82 g of poly(vinyl pyrrolidone) (PVP) (medical grade Kollidone® available in powder form, marketed by BASF, with Mw of 900 000 to 1 200 000 g/mol) were dissolved in 60 ml of 96% concentrated ethanol (in osmosis water i.e. 96% weight/volume) at ambient temperature and with gradual addition to prevent precipitation of the polymer; the mixture was left under gentle agitation at 150 rpm for at least 30 minutes.

Thereafter, about 3 g of bioglass 45S5 were added to the PVP solution and dispersed therein. The concentration of bioglass here was 10% weight/volume of solution.

The solutions were left under agitation for 8h.

### B-Impregnation of a Textile Elongate Core

Advantageously, the textile elongate core comprises an auxiliary core comprising 3 multifilament yarns in polyester (in particular in polyethylene terephthalate) each of 190 dtex, and an auxiliary covering over-braided around said auxiliary core, said auxiliary covering comprising 16 braided strands (i.e. obtained on a loom with 16 spindles all filled), each braided strand comprises a multifilament yarn in polyester (in particular in polyethylene terephthalate) of 90 dtex and a multifilament yarn in polyester (in particular of polyethylene terephthalate) of 230 dtex. Advantageously, the textile elongate core has a mass per unit length of between 0.50 g/metre and 1 g/metre, for example of approximately 0.60 g/metre.

The elongate core was dipped in the solution and then through a mangle device, in this specific example between two rollers rotating at about 2 metre/minute and with the application of a nip pressure of 3 bar. The number of full-bath impregnation and roll passes was about 10.

The elongate core was dried in open air for 24h. In this manner, a first example of elongate core was obtained.

### C-Over-Braiding of an Elongate Covering Member

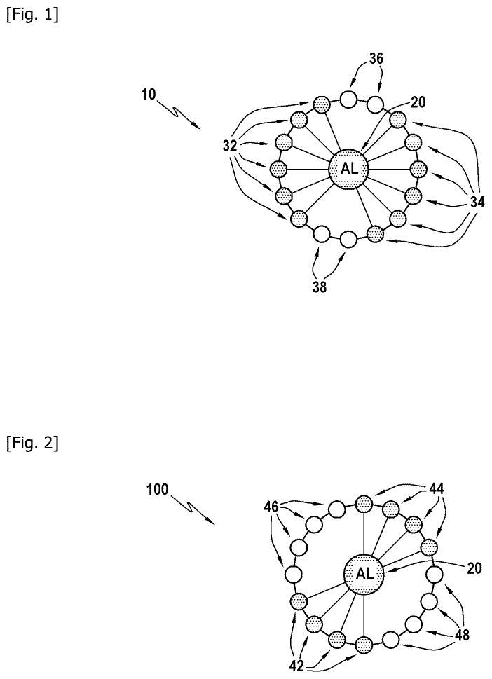

In general, in Examples 1 to 4 below, an empty spindle is represented in FIGS. 1 to 4 by an empty circle, and a filled spindle is represented by dotted circle.

### Example 1 of Implantable Device

A first example of covering member was over-braided, according to the first example of arrangement 10 illustrated in FIG. 1, around the coated elongate core 20 obtained under item B above. FIG. 1 illustrates the arrangement of the spindles of a braiding loom having 16 spindles. In this specific example, a first group of 6 adjacent filled spindles 32 was spaced apart from a second group 6 of adjacent filled spindles by first and second groups 36 and 38 each having empty spindles. Each filled spindle carried 6 multifilament yarns in polyethylene terephthalate each of 138 dtex. In this manner, a first example was obtained of an implantable device of the invention (EX1).

### Example 2 of Implantable Device

A second example of covering member was over-braided, according to the second example of arrangement 100 illustrated in FIG. 2, around the coated elongate core obtained under item B above. FIG. 2 illustrates the arrangement of the spindles of a braiding loom having 16 spindles. In this specific example, a first group of 4 adjacent filled spindles 42 was spaced apart from a second group of 4 adjacent filled spindles 44 by first and second groups 46 and 48 each having 4 empty spindles. Each filled spindled carried 6 multifilament yarns in polyethylene terephthalate of 138 detx each. In this manner, a second example was obtained of an implantable device of the invention (EX2).

### Example 3 of Implantable Device

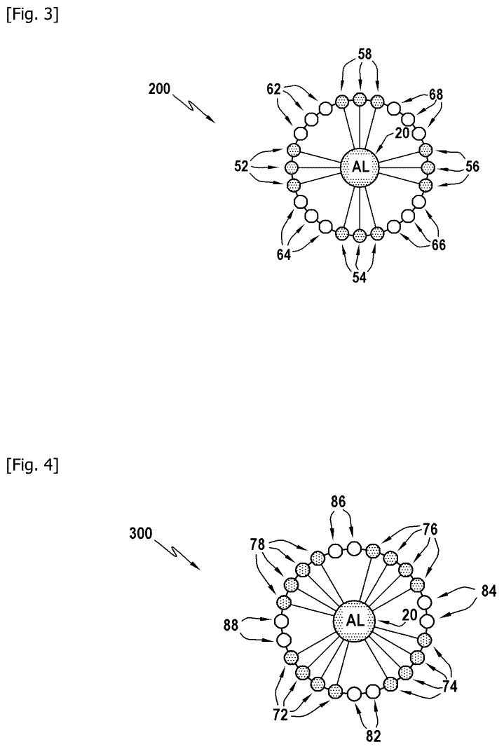

A third example of covering member was over-braided, according to the third example of arrangement 200 illustrated in FIG. 3, around the coated elongate core 20 obtained under item B above. FIG. 3 illustrates the arrangement of the spindles of a braiding loom having 24 spindles. In this specific example, four groups 52, 54, 56 and 58 each of 3 adjacent filled spindles were spaced apart each time by a group of 3 empty spindles 62, 64, 66 and 68. Therefore a group of 3 filled spindles was surrounded by two groups each having 3 empty spindles. Each filled spindle carried 4 multifilament yarns in polyethylene terephthalate each of 138 dtex. In this manner, a third example was obtained of an implantable device of the invention (EX3).

### Example 4 of Implantable Device

A fourth example of covering member was over-braided, according to the fourth example of arrangement 300 illustrated in FIG. 4, around the coated elongate core obtained under item B above. FIG. 4 illustrates the arrangement of the spindles of a braiding loom having 24 spindles. In this specific example, four groups 72, 74,76 and 78 each of 4 adjacent filled spindles were spaced apart each time by a group 82,84,86 and 88 of 2 empty spindles. Therefore, a group 72,74,76 or 78 of 4 filled spindles was surrounded by two groups 82,84,86,88 of 2 empty spindles. Each filled spindle carried 4 multifilament yarns in polyethylene terephthalate each of 138 dtex. In this manner, a fourth example was obtained of an implantable device of the invention (EX4).

### D-Study on Bio-Mineralization

7500 ml of simulated body fluid (SBF) were prepared following the protocol by Tadashi Kokubo and Takadama, Biomaterials 1st May 2006, 27 (15): 2907-15. The reagents were placed in ultrapure water in this order: NaCl (60.2625 g), NaHCO₃ (2.6625 g), KCl (1.6875 g), K₂HPO₄, 3H₂O (1.7325 g), MgCl₂, 6H₂O (2.3325 g), 1 MOL/l of HCl, CaCl₂) (2.19 g), Na₂SO₄ (0.54 g), 293 ml of HCl at 1 Mol/1 was added to adjust pH to 7.40.

At least 3 samples each of length 3 cm for examples EX1, EX2, EX3 and EX4 were immersed in 120 ml of SBF solution following the equation Vs=Sa/10, Vs being the volume of SBF and Sa being the surface area of the sample (mm²).

The incubation times were 1 day (D1), 3 days (D3) and 7 days (D7).

Samples corresponding to EX4 without bioglass were also used as test samples.

The incubation parameters were 80 rpm and the temperature of the SBF solution was held at 37° C. in the agitator. The SBF solution was renewed every 48 hours.

### E-Characterization of the Tested Samples of Examples 1 to 4 (EX1 to EX4)

Morphological and structural analysis of the samples and surface characterization were carried out under scanning electron microscopy (SEM, Hitachi flex 1000) operating at a voltage of 5 kV. The tested samples (EX1 to EX4) were rinsed in distilled water and dried. All the samples were then sprayed with a fine coating of chromium (5 nm thickness) on the outer surfaces thereof. Methodology for evaluation entailed analysing 3 different sites on the outer surface of the covering member, the inner surface of the covering member, as well as the outer surface of the elongate core.

Chemical surface characterization of the samples was performed by Energy Dispersive X-ray Spectroscopy (EDS). Acquisition of diffraction patterns was obtained on a PAnalytical X′PRT Pro MRD diffractometer, with an anode (Cu) as X-ray source (characteristic wavelength of 1.5418 angstrom). The diffractograms were obtained in the range of 5 to 70° (theta) with a detector (X′Celerator®, PAnalytical) equipped with an anti-scatter slit of 0.5° and 1° and 10 mm mask.

### F- In Vitro Study on Cytotoxicity

A cytotoxicity study was performed on examples EX1 to EX4 according to standard ISO 10993-5, 2009. This test proved to be positive i.e. the survival rate of living cells was satisfactory.

### G- Comments

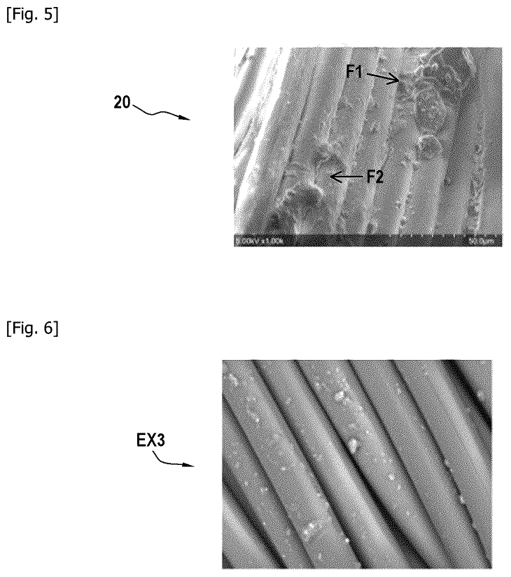

FIG. 5 gives a SEM photograph of the outer surface of the elongate core 20 obtained under item B. The arrows F1 and F2 indicate the formation of bioglass crystals after 7 days' incubation in SBF.

It is observed that after an incubation time of 1 day and 3 days, there is no bio-mineralization on the outer surface of the elongate core 20, on the inner surface or outer surface of the covering member of Examples 1 to 4.

After an incubation time of 7 days, slight bio-mineralization is observed on the outer surface of the covering members of Examples 3 and 4 in the vicinity of the through-holes.

Major bio-mineralization is observed on all the inner surfaces of the covering members of Examples 1 to 4 after 7 days' incubation.

In particular, more numerous bioglass crystals are observed on the outer surface of the covering member of Example 4 illustrated in FIG. 8 than on the outer surface of the covering member of Example 3 illustrated in FIG. 6. However, the through-holes of Examples 3 and 4 have advantageously allowed bio-mineralization of the outer surface of the covering member.

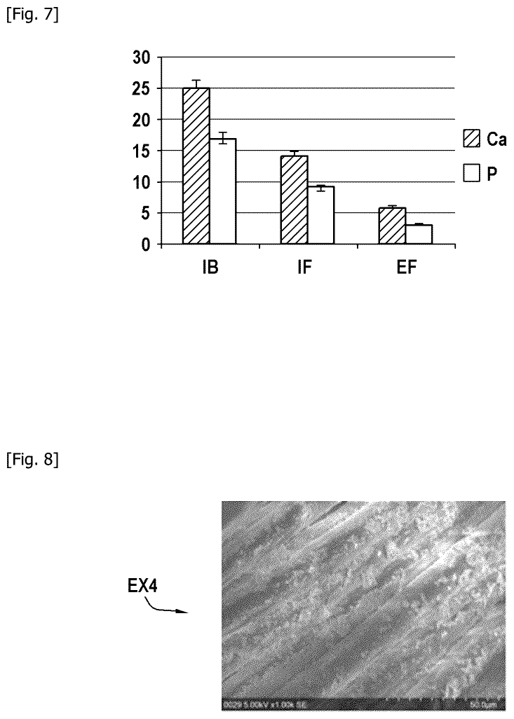

As can be seen in FIG. 7, calcium and phosphorus are abundantly present on the outer surface of the elongate core (IB) and on the inner surface of the covering member (IF) of Example 3. The presence of calcium and phosphorus is also found on the outer surface of the covering member (EF) of Example 3.

By calculating the molar ratio between calcium and phosphorus, a Ca/P ratio of 1.125 is found after 7 days' incubation on the outer surface of the covering member of Example 3, and of 1.75 on the outer surface of the covering member of Example 4.

The molar ratio of hydroxyapatite is estimated to be 1.67.

Finally, more bio-mineralization is observed after an incubation time of 14 days or 21 days for Examples 1 to 4.

Advantageously, the implantable device of the invention ensures the mechanical function of a tendon or ligament, but also allows gradual release of functional agent(s), and particularly allows the formation of hydroxyapatite on the outer surface of the covering member, in particular on and after 7 days for Examples 3 and 4.

1. An implantable device for the reconstruction of an anterior cruciate ligament or tendon, wherein said implantable device comprises an elongate core and a hollow elongate covering member comprising an inside volume at least partially receiving said elongate core, wherein said elongate core comprises a polymer matrix bio-erodible in an aqueous medium and one or more functional agent(s), and wherein the covering member comprises through-holes in fluid communication with said inside volume.

2. The implantable device according to claim 1, wherein the functional agent(s) comprise one or more osteoinductive and/or osteoconductive agents.

3. The implantable device according to claim 2, wherein the osteoinductive and/or osteoconductive agent(s) are chosen from among: bioglass, calcium phosphate-based bioceramics, in particular hydroxyapatites, and tricalcium phosphate, and a mixture thereof.

4. The implantable device according to claim 1, wherein the functional agent(s) are chosen from among: anti-inflammatories, antibiotics, antibacterials, analgesics and a mixture thereof.

5. The implantable device according to claim 1, wherein the elongate core comprises a braided elongate core.

6. The implantable device according to claim 5, wherein the textile elongate core is at least partially coated with the bio-erodible polymer matrix within which the functional agent(s) are dispersed.

7. The implantable device according to claim 1, wherein the hollow elongate covering member comprises a braid elongate hollow covering member.

8. The implantable device according to claim 1, wherein the bio-erodible polymer matrix comprises a erodible polymer or several mixed erodible polymers that are dehydrated and able to form a soluble gel in an aqueous medium.

9. The implantable device according to claim 1, wherein the bio-erodible polymer matrix comprises one or more bio-erodible polymers chosen from among the following polymers: polymers of cyclodextrin(s) and/or of derivative(s) of cyclodextrin(s) and/or of inclusion complex(es) of cyclodextrin(s) and/or of derivatives of soluble cyclodextrin(s); collagen polymers; polymers derived from hyaluronic acid; polymers of carboxymethylcellulose or derivatives of carboxymethylcellulose; vinyl polymers; polyvinylpyrrolidone polymers; polyvinylpolypyrrolidone polymers; polyethylene glycol polymers; lactic acid polymers; copolymers of lactic acid and polyethylenegylcol; copolymers of lactic acid, glycolic acid and polyethyleneglycol; polymers of polyvinyl alcohols; and polymers of polyacrylates; polyhydroxyethylmethacylate; and a mixture thereof.

10. The implantable device according to claim 1, wherein the bio-erodible polymer matrix comprises a polymer chosen from among: polyvinylpyrrolidone polymers; polyvinyl alcohol polymers; polymers of cyclodextrin(s) and/or derivative(s) of cyclodextrin(s) and/or of inclusion complex(es) of cyclodextrin(s) and/or of derivative(s) of soluble cyclodextrin(s).

11. The implantable device according to claim 1, wherein the bio-erodible polymer matrix comprises one or more bio-erodible polymer(s), and wherein the weight ratio of function agent(s)/bio-erodible polymer(s) in the polymer matrix is higher than or equal to 0.2 and lower than or equal to 1.8.

12. The implantable device according to claim 1, wherein the hollow elongate covering member comprises a braid comprising between 8 and 30 braided strands, and wherein each braided strand comprises between 2 and 8 yarns.

13. The implantable device according to claim 1, wherein the hollow elongate covering member comprises between 30 and 120 yarns that are not bioresorbable.

14. The implantable device according to claim 1, wherein the mass fraction of the bio-erodible polymer matrix comprising the functional agent(s) in said implantable device is higher than or equal to 5% and lower than or equal to 25%.

15. The implantable device according to claim 1, wherein the mass fraction of the elongate core free of the polymer matrix, of said functional agent(s) and of said hollow elongate covering member, measured relative to the total mass of the implantable device, is higher than or equal to 75%.

16. The implantable device according to claim 1, wherein the hollow elongate covering member is a hollow braid with a substantially constant braiding angle greater than or equal to 2 mm and smaller than or equal to 15 mm.

17. A method for manufacturing an implantable device wherein said method comprises:
a) a step to apply a bio-erodible polymer coating comprising one or more functional agent(s) onto an elongate textile, core
b) a step to arrange by over-braiding a hollow elongate covering member around at least part of said at least partially coated elongate core;
c) a step to recover an implantable device comprising a textile elongate core and a braided hollow elongate covering member having an inside volume at least partially receiving said textile elongate core, said textile elongate core comprises a polymer matrix bio-erodible in an aqueous medium and one or more functional agent(s), and the covering member comprises through-holes in fluid communication with said inside volume.

18. The manufacturing method according to claim 17, wherein step b) is a step to braid a hollow braid on a braiding device comprising a total number of spindles higher than or equal to 12 and lower than or equal to 30, and wherein the number of empty spindles.

19. The manufacturing method according to claim 17, wherein step b) is a braiding step of a hollow braid on a braiding device comprising filled spindles and empty spindles, and wherein the filled spindles are distributed in at least three groups of filled spindles in which each group comprises adjacent filled spindles, and wherein a group of filled spindles is separated from another group of filled spindles by at least one empty spindle.

20. The manufacturing method according to claim 19, wherein the braiding device comprises three or four groups of three or four adjacent filed spindles, and three or four groups of adjacent empty spindles, a group of adjacent filled spindles being arranged between two groups of adjacent empty spindles.

21.-22. (canceled)
